(19) United States
(12) Patent Application Publication
(10) Pub. No.: US 2008/0140034 A1
(43) Pub. Date: Jun. 12, 2008

(54) Women's urinary (or urostomy) leg bag with adjustable elastic waist and thigh straps

(76) Inventor: Areta Bullock Edling, McKinney, TX (US)

Correspondence Address: ARETA EDLING, 2314 NORTH RIDGE RD, MCKINNEY, TX, 75070, US

(21) Appl. No.: 11/634,759

(22) Filed: Dec. 6, 2006

(51) Int. Cl. A61M 1/00

(52) U.S. Cl. 604/327

(57) ABSTRACT

The present invention allows for drainage downward with gravity force but not so low that it prevents women from being able to wear comfortable and fashionable clothing. The present invention allows women to have the same medical results as men who need to wear urinary ostomies (or urostomies), or urinary leg bags, but with the urine pouch being placed higher on the leg, or right above the knee or mid-thigh. The present invention is distinguishable for conventional products in that it has a pocket that the holder slips into and has a hole designed into the bottom of the leg bag holder for which to insert the drainage valve.

### TECHNICAL FIELD

The present invention relates to urinary (or urostomy) leg bags.

### BACKGROUND OF THE INVENTION

There are various physical or medical reasons that cause bladder incontinence with the need for ostomies or supra pubic catheter placement. These medical conditions include neurogenic bladders (where the bladder has nerve damage coming from the spine causing bladder damage); or because of chemotherapy or radiation therapy, or other kinds of cancers; frequent urinary tract or kidney infections (UTIs), and many other medical conditions causing the bladder not to empty properly. When these kinds of problems occur, it is necessary to medically place a supra pubic catheter right above the pubic bone, permanently into the bladder through a hole in the outside of the body which allows the bladder to constantly drain into a leg bag or pouch. Conventionally, these products are fitted below the knee with a drainage tube that goes all the way down the leg and fits onto a line that allows the urine to fill into a pouch. Most of the “drainage” type products are invented for men, or considered “unisex”. If women use these products they have to wear long dresses (to the ankles) or loose leg slacks. This limits attire that women can purchase or make for themselves and still stay somewhat fashionable and comfortable.

Conventional products include: Aquasleeve Leg Bag Support, a sleeve that fits around the lower leg; Bard Polyester Leg Bag Holder, a sleeve that fits around the lower leg; IIMedico One Leg Pant, one leg that goes primarily from the waist to the knee and must be worn on the left leg; Rusch Belly Drain Bag, an apparatus that attaches to the front of the catheter for drainage; Afex urine collection system which appears to be designed something like athletic-style boxer briefs and BetterPant by External Collection Systems (RCS), which appears to look like biker shorts with a front opening with an attaching sheath.

What is desired is a urinary (or urostomy) leg bag holder that allows for drainage downward with gravity force but not so low that it prevents women from being able to wear comfortable and fashionable clothing.

### SUMMARY OF THE INVENTION

The present invention allows for drainage downward with gravity force but not so low that it prevents women from being able to wear comfortable and fashionable clothing. The present invention allows women to have the same medical results as men who need to wear urinary ostomies (or urostomies) or urinary leg bags, but with the urine pouch being placed higher on the leg, or right above the knee or mid thigh. The present invention is distinguishable from conventional products in that it has a pocket that the holder slips into and has a hole designed into the bottom of the leg bag holder through which to insert the drainage valve

To those skilled in the art to which this invention relates, many changes in construction and widely differing embodiments and applications of the invention will suggest themselves without departing from the scope of the invention as defined in the appended claim. The disclosures and the descriptions herein are purely illustrative and are not intended to be in any sense limiting.

### BRIEF DESCRIPTION OF THE DRAWINGS

For a better understanding of the present invention including the features, advantages and specific embodiments, reference is made to the attached drawings. The physical structure of the present invention is shown in shown in the following Figures.

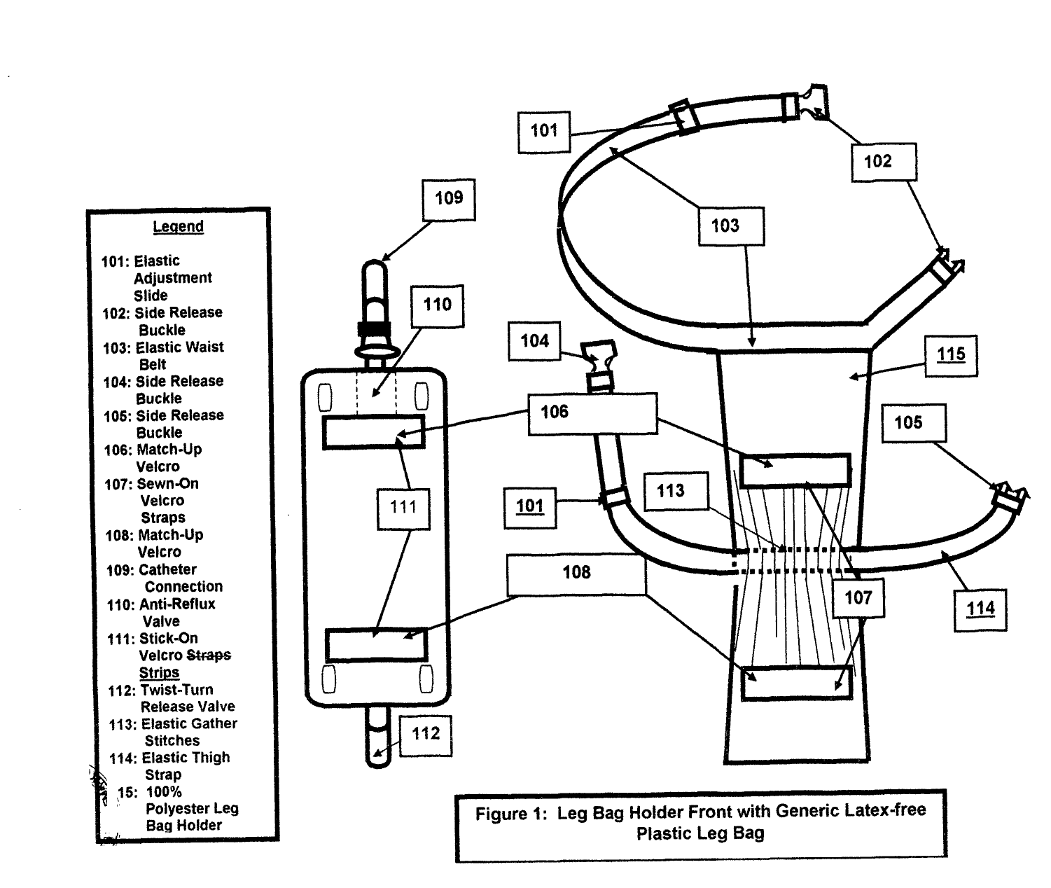

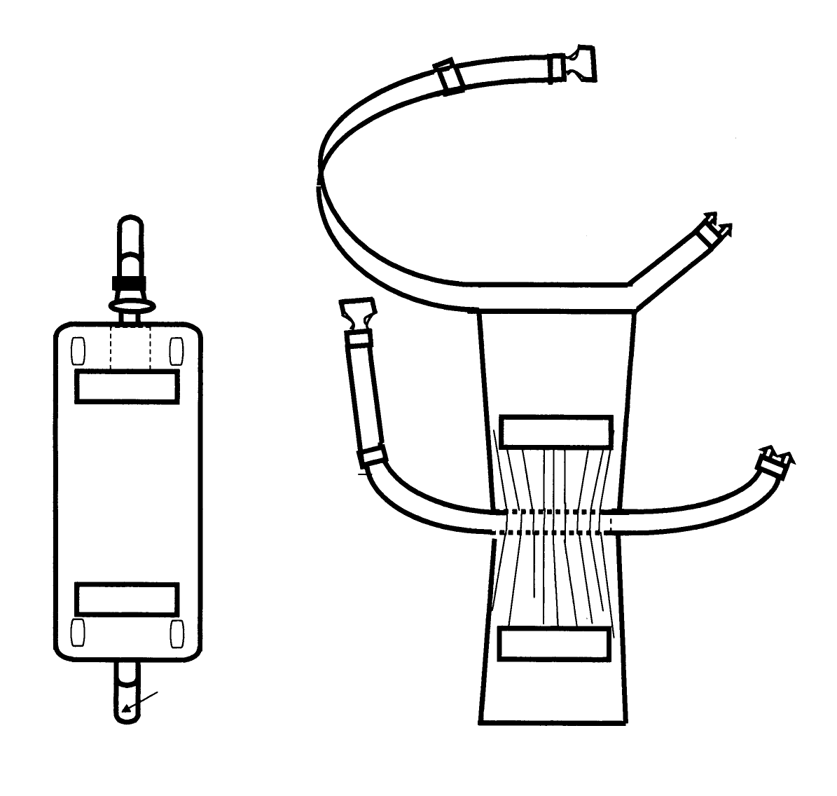

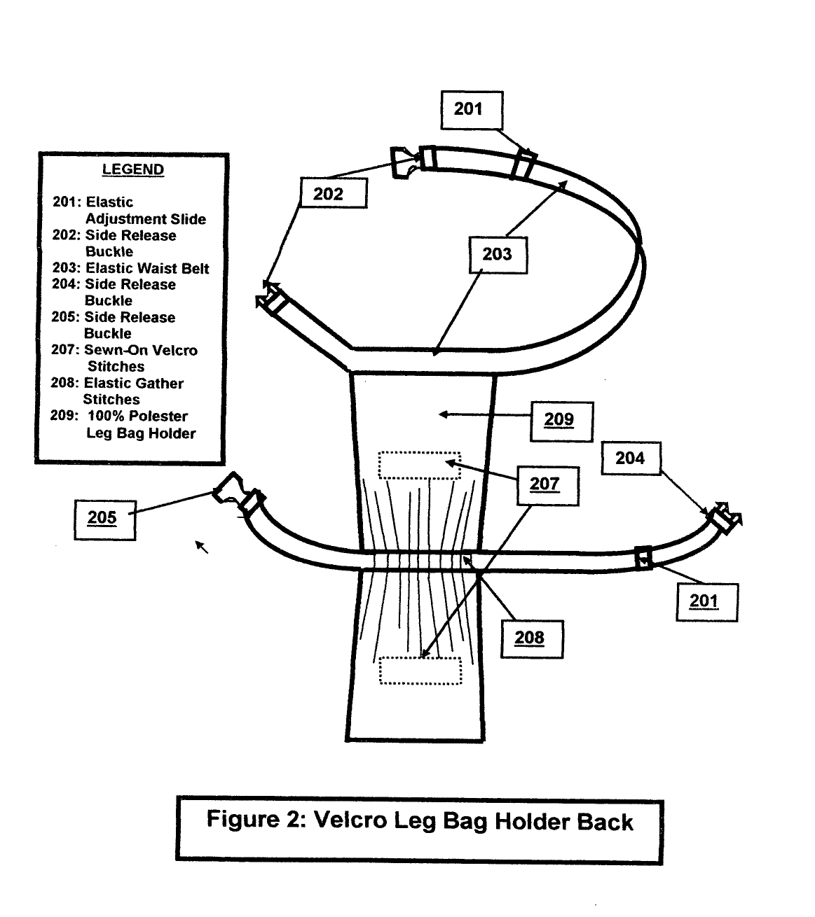

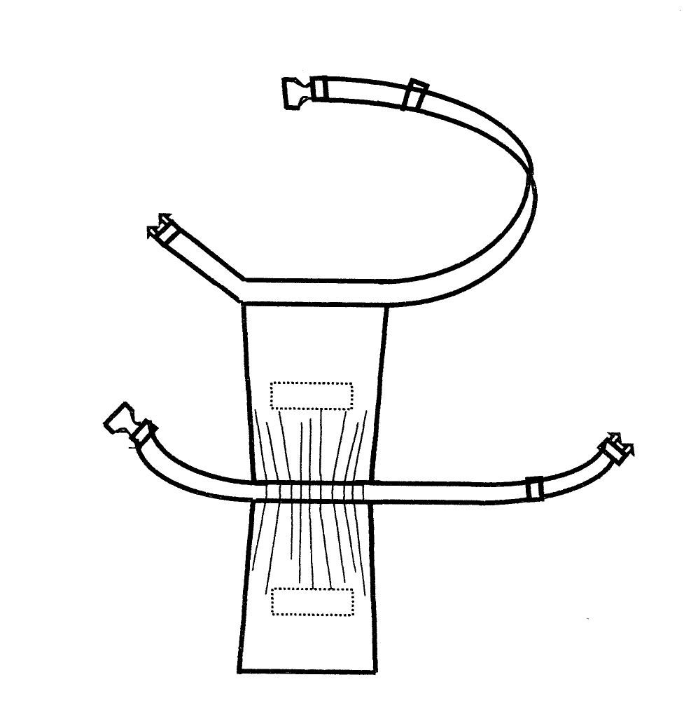

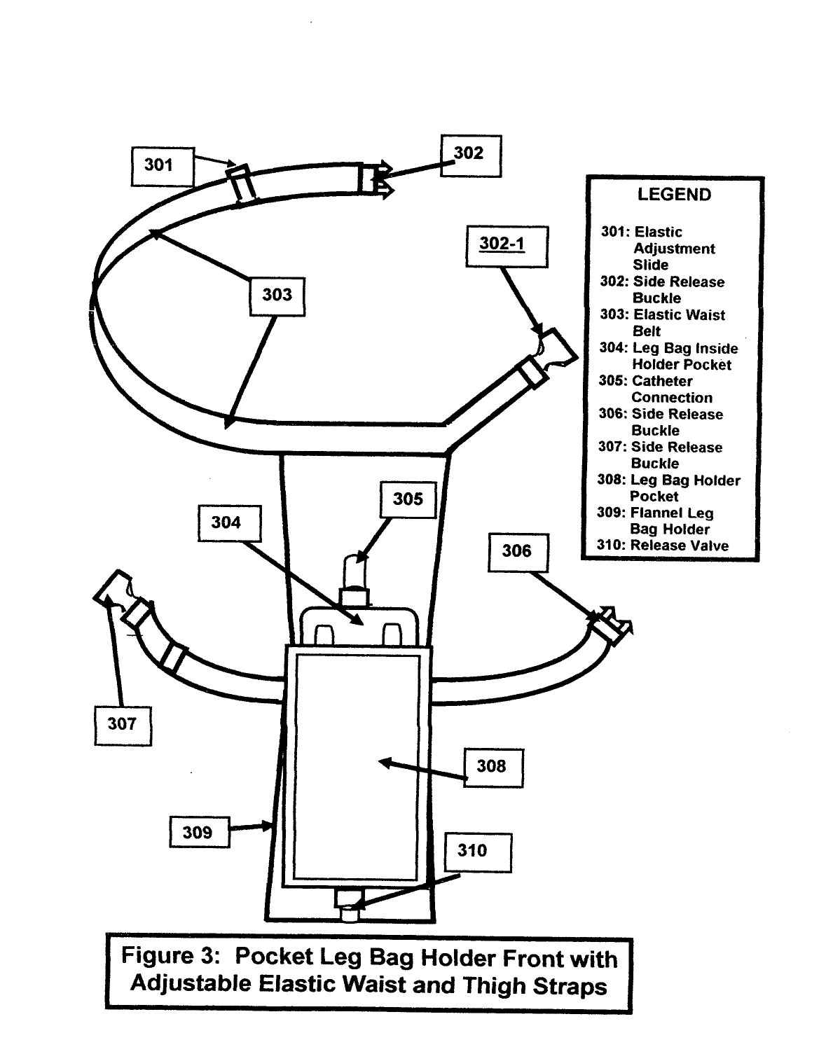

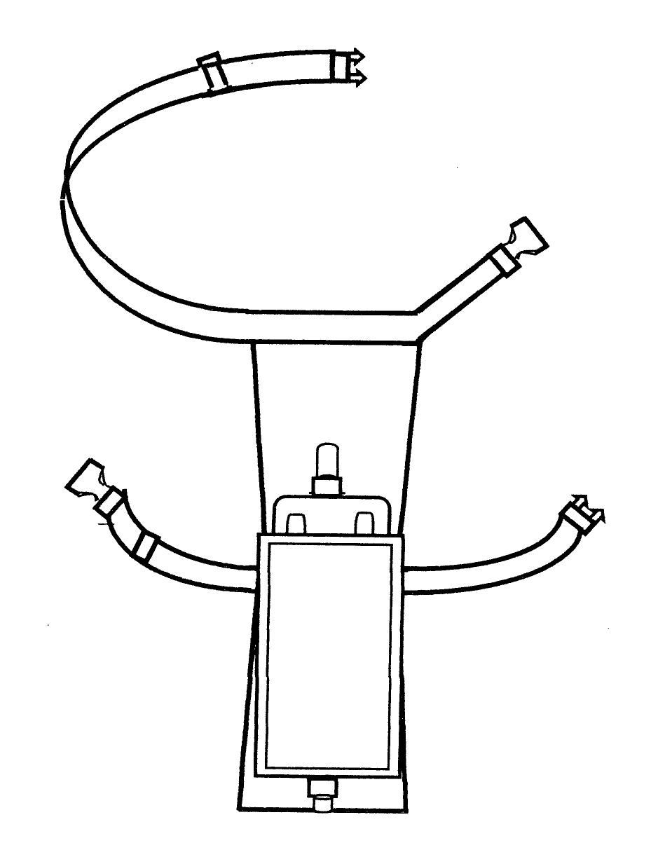

FIG. 3 is the full front view of the second embodiment Pocket Leg Bag.Holder with Adjustable Elastic Waist and Thigh Straps. It is similar to the first embodiment in that 309 is the part of the leg bag holder that is made from two-ply 100% polyester flannel. The holder hangs from the waist, is approximately 6″ to 8″ wide and 17″ to 21″ long depending on the customer's preferred size. Sizes range from small, medium, large and extra large. It can also be supplied in special order sizes by request from customer.
301 Is the front side of the elastic adjustment slide (identical to the elastic adjustment slide from 101 and 201 from FIG. 1 of first embodiment.) It is an elastic size adjustment for both the waist 1″ non-roll elastic as well as the thigh 1″ non-roll elastic. 302 and 302-1, as well as 306 and 307(each) are the two independent ends of the side release white plastic elastic buckle at the ends of the waist and thigh elastic. These two ends match up and snap together when attached around the thigh thereby making the buckle 1″×3″ when attached together.
303 is the front side of the 1″ non-roll elastic at the waist.
   304 shows the leg bag inside the holder pocket;
   305 is the leg bag catheter connection: and
   310 is the leg bag release valve for release of urine. (304, 305 and 310 are depicted for effect only and are not included in the invention).
308 is the pocket in which the leg bag is placed for the second embodiment. It is constructed from 100% two ply polyester flannel as is the rest of the lag bag holder.
309 is the leg bag holder that is made from two-ply 100% polyester flannel. The holder hangs from the waist, is approximately 6″ to 8″ wide and 17″ to 21″ long depending on required size. Sizes range from small, medium, large and extra large. It can also be supplied in special order sizes by request from customer.

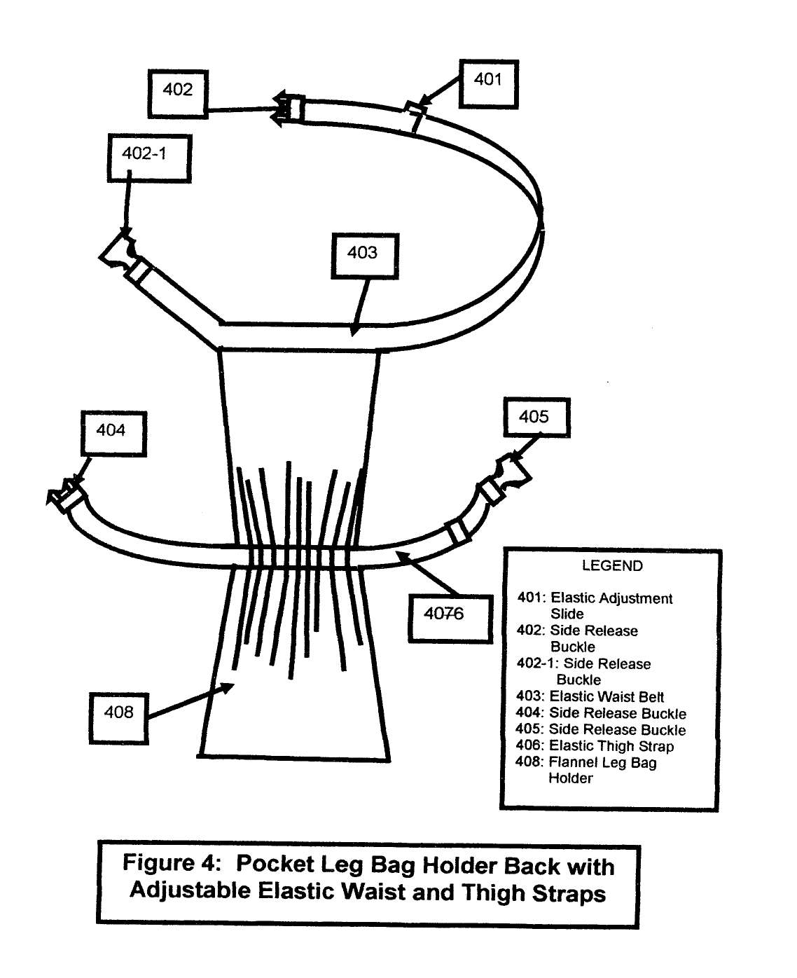

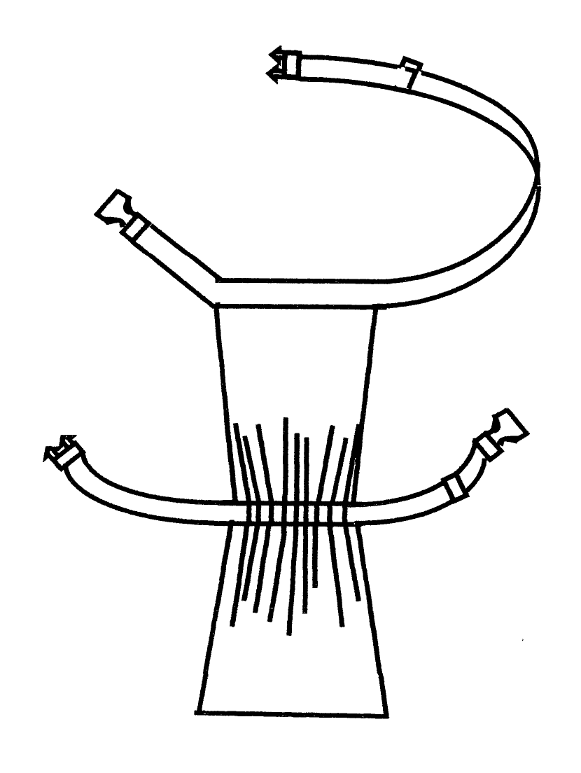

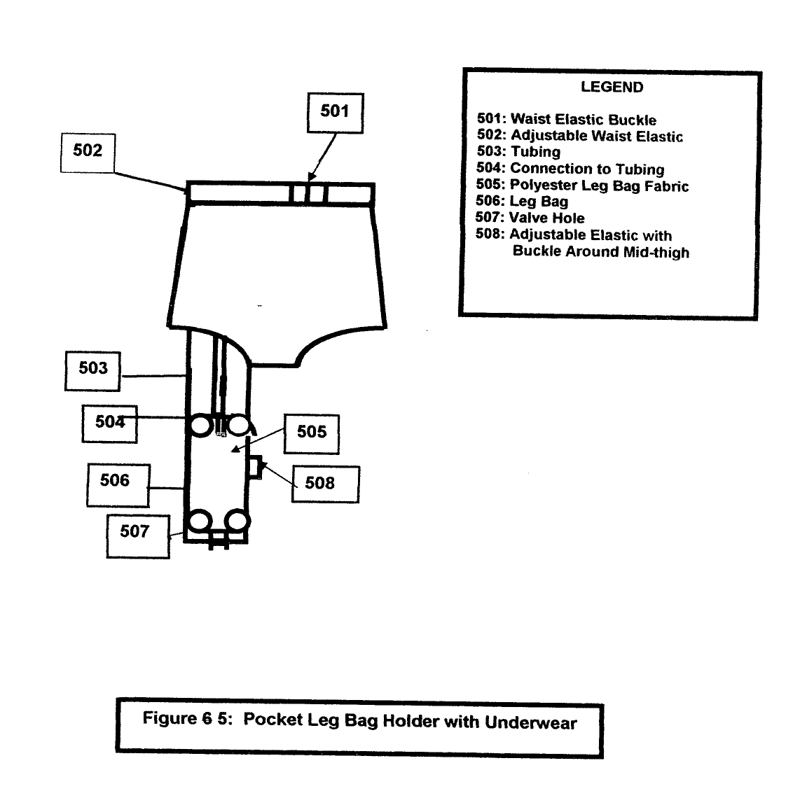

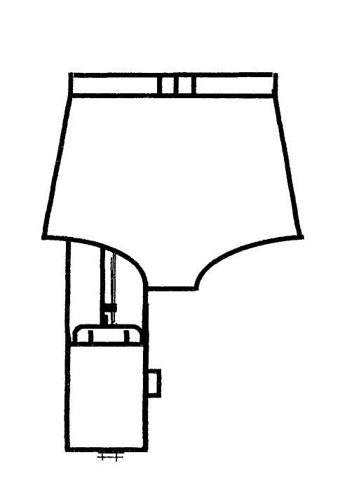

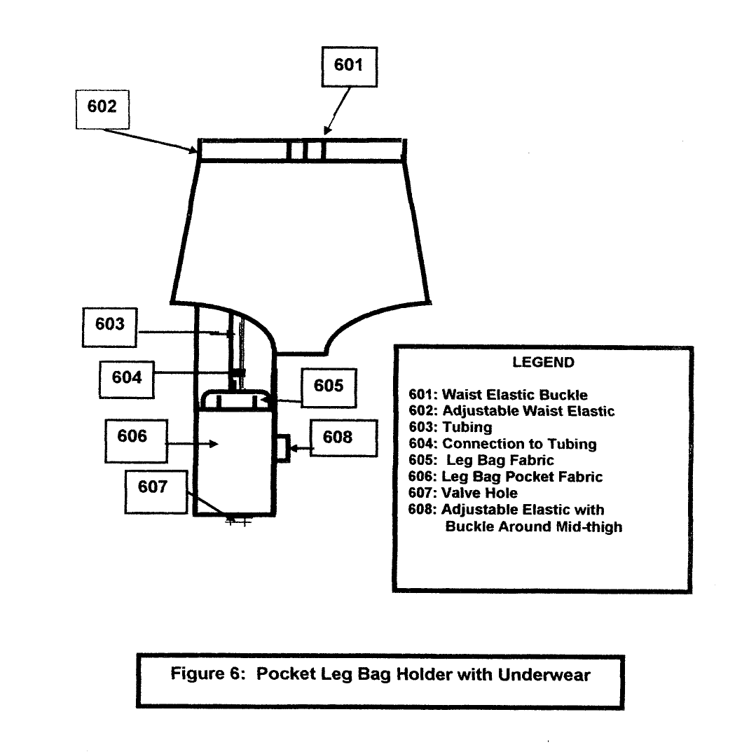

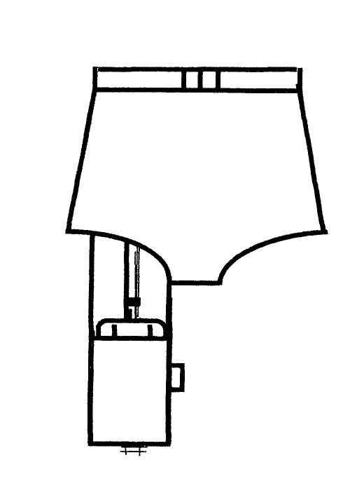

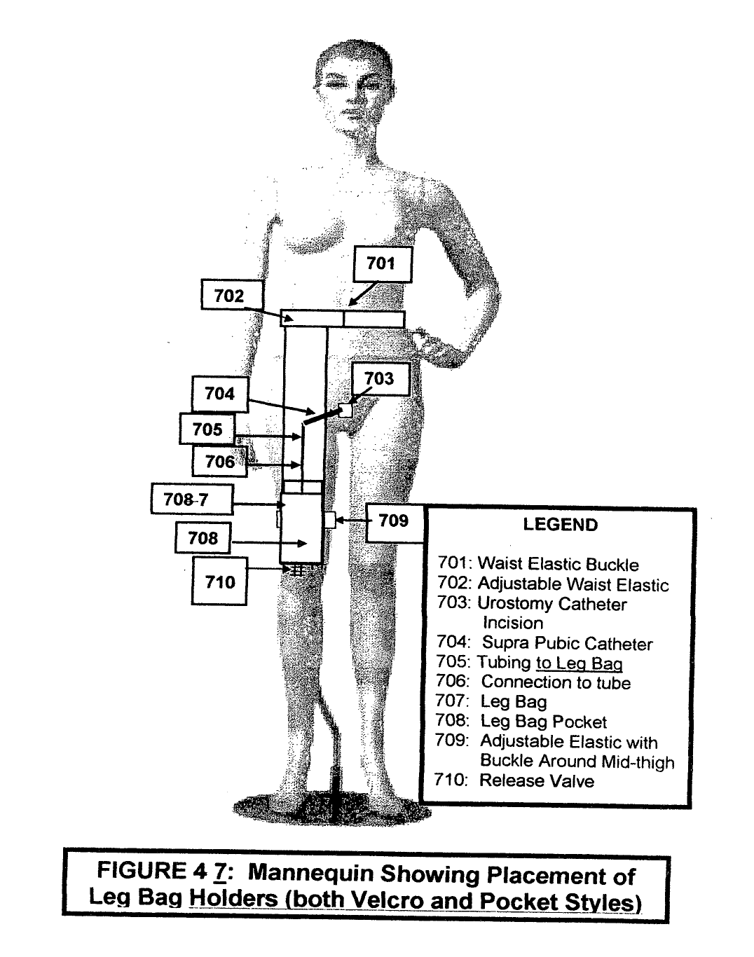

FIG. 4 is the Pocket Leg Bag Holder Back with Adjustable Waist and Thigh Straps.
401 Is the front side of the elastic adjustment slide (identical to the elastic adjustment slide from 101 and 201 from FIG. 1 of First Embodiment, as well as 301 from the Second Embodiment). It is an elastic adjustment for sizing both the waist 1″ non-roll elastic as well as the thigh 1″ non-roll elastic. 402, 402-1, 404 and 405 are side release buckles
403 is the 1″ non-roll elastic waist belt.
406 is the 1″ non-roll elastic thigh strap.
408 is the leg bag holder that is made from two-ply 100% polyester flannel. The holder hangs from the waist, is approximately 6″ to 8″ wide and 17″ to 21″ long depending on required size. Sizes range from small, medium, large and extra large. It can also be supplied in special order sizes, by request, from customer.

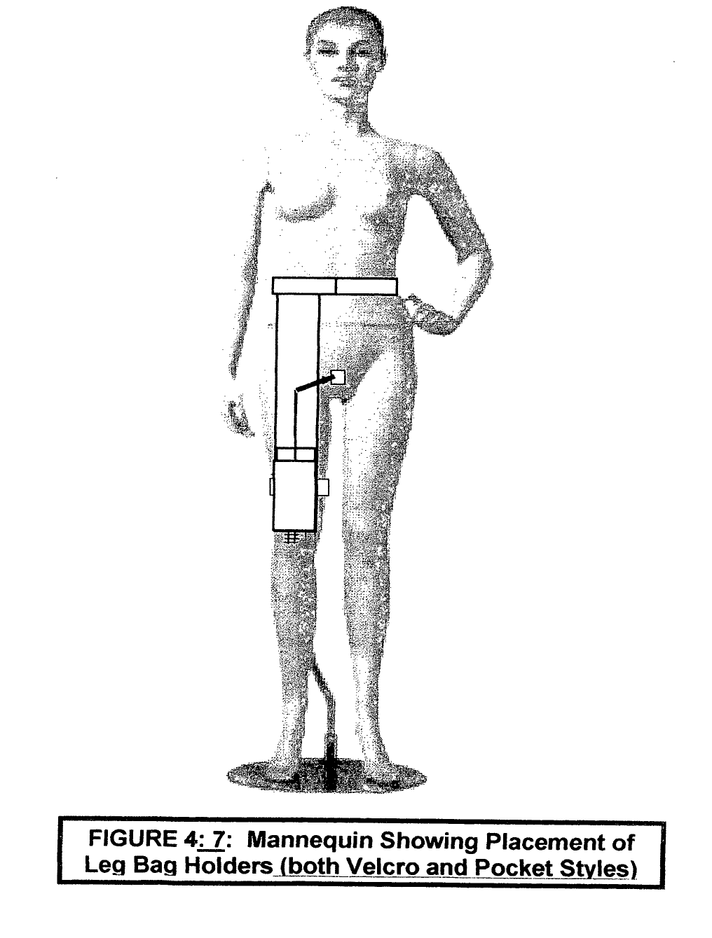

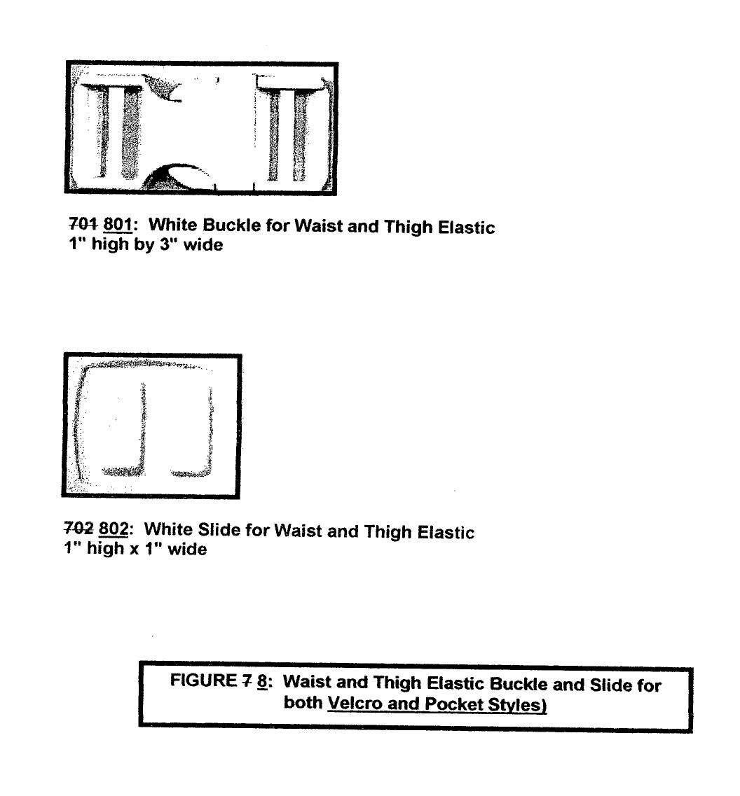

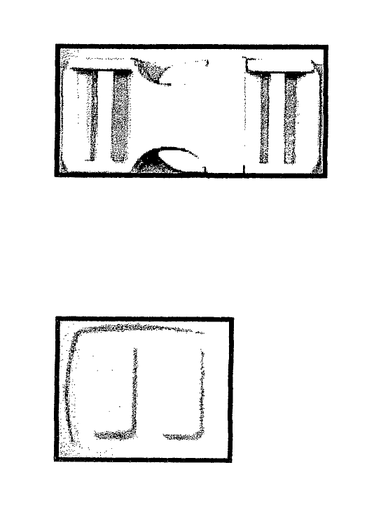

FIG. 6 depicts The Women's Urinary Pocket Leg Bag Holder with Adjustable Waist and Thigh Straps being worn under underwear.
601 is the white elastic buckle for the waist.
602 is the adjustable 1″ wide non-roll waist elastic.
603, 604, 605, 606, 607 are parts of the plastic latex leg bag.
608 is the adjustable buckle around the mid-thigh.

### DETAILED DESCRIPTION OF A PREFERRED EMBODIMENT

While the making and using of the preferred embodiment of the present invention is discussed in detail below, it should be appreciated that the present invention provides many applicable inventive concepts that can be embodied in a wide variety of specific contexts. Some features of the preferred embodiment shown and discussed may be simplified or exaggerated for illustrating the principles of the invention.

The present invention allows women who have the same medical condition as men who need to wear urinary ostomies (or urostomy) leg bags, but with the urine pouch being placed higher on the leg, or right above the knee or mid thigh, and attached to the holder using Velcro straps.

The present invention comprises a strip of soft flannel fabric about 6″ to 8″ wide (depending on the customer's preferred size of garment) that hangs from a 1″ adjustable non-roll elastic “belt” and has a 1″ wide strip of elastic to hold the leg bag holder in place around mid thigh and has a pocket holder adapted to hold the leg bag in the holder. The adjustable non-roll 1″ elastic is placed around the waist and mid-thigh, and the holder comes in sizes small, medium, large and extra large. Special orders for larger sizes will also be done at an additional cost. The garment is basically made to fit on the right leg, but with the customer's request, it can be made to be fit on the left leg at no additional cost. With the elastic being further adjustable it will fit women in in-between sizes as well. It is to be worn under undergarments. The present invention comes in white.

The present invention is adapted to allow the leg bag holder to be fit as a finished piece of 100% polyester flannel fabric with a pocket above the knee and therefore allows women to wear more fashionable clothing while attaining the same results and that being downward flow of urine.

The present invention functions as a holder that hangs from an elastic belt and the leg bag fits into a bag sewn onto the front of the holder. The present invention will not interfere if the wearer has to also wear diapers or other ostomy garments, or if the person is in a wheel chair. Neither will it show under clothing, even fairly tight fitting slacks or skirts.

The present invention is specifically for women to be worn under women's clothing for a more cosmetic appearance but allows for the downward flow of gravity flow of urine into the leg bag. The present invention also allows for the free flow of air thereby making it more comfortable during the warm or hot months of the year or for those who live in warm climates the year round.

Women who need to wear urinary ostomies (or urostomies) or have supra pubic catheters, but like for their clothing to be current styles and thereby not have to always wear loose leg pants or long-to-the ankle dresses or skirts in order to disguise the leg bag and holder.

The embodiments shown and described above are only exemplary. Even though numerous characteristics and advantages of the present invention have been set forth in the foregoing description together with details of the method of the invention, the disclosure is illustrative only and changes may be made within the principles of the invention to the full extent indicated by the broad general meaning of the terms used in the attached claims.

I claim:

1. A urinary (or urostomy) leg bag holder, comprising:
a strip of soft 100% polyester flannel fabric about 6″ to 8″ wide (depending on the customer's preferred size of the garment) that hangs from a 1″ adjustable non-roll elastic “belt” and has a 1″ wide strip of elastic to hold the leg bag holder in place around mid thigh:
a pocket holder adapted to hold the leg bag in the holder
an adjustable non-roll 1″ elastic strip is placed around the waist and mid-thigh, and the holder is available in sizes small, medium, large, and extra-large; and
the elastic being adjustable to fit various sizes of women.
